(19) United States
(12) Patent Application Publication
(10) Pub. No.: US 2009/0162404 A1
(43) Pub. Date: Jun. 25, 2009

(54) Tumor vaccine

(76) Inventor: Eckhard R. Podack, Coconut Grove, FL (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 10/950,157

(22) Filed: Sep. 24, 2004

Related U.S. Application Data
(60) Provisional application No. 60/506,656, filed on Sep. 26, 2003.

(51) Int. Cl. A61K 39/00

(52) U.S. Cl. 424/277.1

(57) ABSTRACT

The invention provides a tumor cell genetically modified to express a nucleic acid encoding CD80 (B7.1) and a nucleic acid encoding an HLA antigen. The invention also provides a method of stimulating an immune response to a tumor by administering an allogeneic tumor cell such as a lung cancer cell genetically modified to express a nucleic acid encoding CD80 (B7.1) and a nucleic acid encoding an HLA antigen. The invention additionally provides a method of inhibiting a tumor by administering an allogeneic tumor cell such as a lung cancer cell genetically modified to express a nucleic acid encoding CD80 (B7.1) and a nucleic acid encoding an HLA antigen.

This application claims the benefit of priority of U.S. Provisional Application Ser. No. 60/506,656, filed Sep. 26, 2003, the entire contents of which is incorporated herein by reference.

This invention was made with United States government support under grant number CA39201-14 awarded by the National Institutes of Health. The United States Government may have certain rights in the invention.

### FIELD OF THE INVENTION

The invention relates to the fields of medicine, immunology, and oncology. More specifically, the invention relates to methods and compositions for inducing an immune response against a tumor in an animal subject.

### BACKGROUND OF THE INVENTION

Lung cancer is the most common cause of death due to cancer in the United States. For 2002, the American Cancer Society predicted that almost 170,000 new cases of lung cancer would be diagnosed and that 155,000 people would die from the disease. Patients with locally advanced or metastatic non-small cell lung cancer (NSCLC) make up 70% of the newly diagnosed cases.

Current recommendations for patients with inoperable disease include platinum-based chemotherapy plus radiation therapy in locally advanced disease, or chemotherapy alone in patients with metastases. Typical response rates are between 15% to 30%, with median survivals of less than one year. Meta-analysis of 52 phase III clinical trials randomizing metastatic NSCLC patients between best supportive care and chemotherapy concluded that chemotherapy increases the chance of 1 year survival by 10% and the median survival by 6 weeks. A recent report from the Big Lung Trial group (BLT) reported similar results. The aggressiveness of NSCLC is thought to relate to its ability to evade the immune system perhaps by suppressing immune response priming by means of CD4 regulatory cells and/or by producing immunosuppressive cytokines such as TGF-β.

Thus, there exists the need to develop effective therapies to treat a tumor, including cancers such as lung cancer. The present invention satisfies this need and provides related advantages as well.

### SUMMARY

The invention provides a tumor cell, for example, a lung cancer cell or other tumor cells, genetically modified to express a nucleic acid encoding CD80 (B7.1) and a nucleic acid encoding an HLA antigen. The invention also provides a method of stimulating an immune response to a tumor, including a cancer tumor such as a lung cancer tumor, by administering an allogeneic lung cancer tumor cell genetically modified to express a nucleic acid encoding CD80 (B7.1) and a nucleic acid encoding an HLA antigen. The invention additionally provides a method of inhibiting a tumor, including a cancer such as lung cancer, by administering an allogeneic tumor cell, for example a cancer tumor cell such as a lung cancer tumor cell, genetically modified to express a nucleic acid encoding CD80 (B7.1) and a nucleic acid encoding an HLA antigen.

### BRIEF DESCRIPTION OF THE DRAWINGS

FIGS. 1A and B shows flow cytometry analysis. Panel A: Quality control of vaccine cells. Representative samples of vaccine cells coexpressing B7.1 (CD80) and HLA A1 (left panel) or HLA A2 (right panel) analyzed by flow cytometry. The percentage of double positive cells is indicated. CD80 and the HLA A allele must be coexpressed on 70% or more of the cells to qualify for immunization. Panel B. Patient CD8 cells purified for ELI-spot assays. Flow cytometry of a representative sample of patient CD8 (right panel) cells purified by negative selection and used for ELIspot analysis; the purity of cells is given in %. Left panel shows isotype control.

FIG. 2 shows analysis of CD8 immune response: Immunization of advanced lung tumor patients generates strong CD8 response. The frequency of IFN-γ-spot forming CD8 cells obtained from lung tumor patients is plotted against the time on study in weeks. Immunizations were given every two weeks, zero representing the preimmunization status. 20,000 purified CD8 cells were used for ELI-spot assays. Panel A: Frequency of spot forming CD8 cells from HLA A1 and A2 positive patients challenged with HLA A1 or A2 transfected (matched) AD100 tumor cells at a ratio of 20:1=CD8:AD100. Panel B: Frequency of spot forming CD8 cells from HLA A1 positive patients challenged with A2-AD100 or HLA A2-CD8 cells were challenged with A1-AD100 (mismatched). Panel C: Frequency of spot forming CD8 cells from non HLA A-1 or A2 patients cells challenged with A1 and A2 transfected AD100 (unmatched). Panel D: Frequency of spot forming CD8 cells from all patients challenged with untransfected w.t. AD100 or, Panel E, with K562. Panel F: Mean frequency of spot forming CD8 cells from all patients challenged with any of the AD100 w.t. or transfected cells. Panel G: CD8 spot forming response of individual, clinically responding patients. The mean number of spots after restimulation with AD100 w.t., AD100-A1, AD100-A2, K562 or nothing in quadruplicate wells is plotted against time after study entry. Arrows indicate the time of last immunization. Patient 1004, 1007, 1010 contain follow up data analyzed at the points indicated after completion of nine immunizations (18 weeks). HLA type of each patient is indicated in brackets.

FIG. 3 shows the median survival time of all patients at the time of analysis. The median survival time was 18 months, exceeding the expected median survival time of less than one year for this group of patients.

FIG. 4 shows overall survival for the 19 B7 vaccine-treated non-small-cell lung cancer study patients.

FIGS. 5A and B show analysis of CD8 immune response. FIG. 5A (top two panels) shows CD8 prior to immunization or at 6, 12 and 18 weeks after challenge with untransfected (AD wild type) vaccine cells or K562 control. FIG. 5B (lower six panels) shows CD8 response after termination of vaccination (arrow) in patients with clinical response.

### DETAILED DESCRIPTION

The invention relates to the discovery that administering allogeneic tumor cells expressing or caused to express CD80 (B7.1) and HLA antigens to cancer patients resulted in an anti-tumor immune response in the patients. More particularly, CD8-mediated immune responses were elicited in stage IIIB/IV NSCLC patients immunized several times with allogeneic NSCLC cells transfected with CD80 (B7.1) and HLA-A1 or A2. Immunization significantly increased the frequencies of interferon-γ-secreting CD8 T cells in all but one of the patients tested as discussed in more details (infra). In a clinical analysis of one set of patients, five of fourteen patients responded to immunization with stable disease or partial tumor regression. Further characterization was performed with additional patients.

Carcinoma of the lung is the leading cause of cancer death and the second most commonly occurring cancer in both men and women in the United States (Jemal, et al., CA Cancer J. Clin. 53:5-43 (2003). Non-small-cell lung cancers (NSCLC) are considered to be minimally or nonimmunogenic, and may contain CD4 regulatory cells that suppress generation of cytotoxic lymphocytes (CTL) (Woo, et al., J. Immunol. 168:4272-4276 (2002)). Although NSCLC has not been considered a good candidate for immunotherapy, the studies disclosed herein are based on the hypothesis that NSCLC is indeed suitable for successful vaccine therapy because the tumor cells have not been exposed to immune attack and have not yet developed resistance mechanisms. Immunotherapy trials for lung cancer have previously yielded no consistent benefit in humans (Ratto, et al., Cancer 78:244-251 (1996); Lissoni, et al., Tumori 80:464-467 (1994); Ratto, et al., J. Immunother 23:161-167 (2000)). Vaccine trials with B7.1 (CD80) transfected allogeneic or autologous cells have not been reported in patients with NSCLC prior to the studies disclosed herein, although similar vaccines have shown good activity in other human studies (Antonia, et al. J. Urol. 167:1995-2000 (2002); Horig, et al, Cancer Immunol. Immunother. 49:504-514 (2000); Hull, et al., Clin. Cancer. Res. 6:4101-4109 (2000); von Mehren, et al., Clin. Cancer Res. 6:2219-2228 (2000)). The objectives of the studies disclosed herein were to assess the safety, immunogenicity, and clinical response to an allogeneic whole cell tumor vaccine transfected with CD80 and HLA A1 or A2 administered to patients with advanced metastatic NSCLC. Disclosed herein are results on vaccine safety, clinical response, and overall survival.

As disclosed herein, to determine whether CD8 mediated immune responses could be elicited in stage IIIB/IV NSCLC patients, initially fourteen subjects were immunized several times with allogeneic NSCLC cells transfected with CD80 (B7.1) and HLA-A1 or A2. Additional patients were added. Patients enrolled were matched or unmatched at the HLA A1 or A2 locus and their immune response compared. Immunization significantly increased the frequencies of interferon-γ secreting CD8 T cells in all but one patient in response to ex vivo challenge with NSCLC cells. The CD8 response of matched and unmatched patients was not statistically different. NSCLC reactive CD8 cells did not react to K562. Clinically, five of fourteen patients responded to immunization with stable disease or partial tumor regression. The study demonstrates that CD8 IFN-γ responses against non-immunogenic or immunosuppressive tumors can be evoked by cellular vaccines even at advanced stages of disease. The positive clinical outcome suggests that non immunogenic tumors may be highly susceptible to immune effector cells generated by immunization.

Thus, it has been discovered that the administration to a tumor patient of modified tumor cells expressing CD80 and an HLA antigen results in desirable therapeutic effects. Hence, in one embodiment, the invention provides a tumor lung cancer cell into which has been introduced a first nucleic acid encoding CD80 and a second nucleic acid encoding HLA antigen.

As used in this specification, the singular forms “a,” “an” and “the” specifically also encompass the plural forms of the terms to which they refer, unless the content clearly dictates otherwise. As used herein, unless specifically indicated otherwise, the word “or” is used in the “inclusive” sense of “and/or” and not the “exclusive” sense of “either/or.” In the specification and the appended claims, the singular forms include plural referents unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “tumor” is used to denote neoplastic growth which may be benign (e.g., a tumor which does not form metastases and destroy adjacent normal tissue) or malignant/cancer (e.g., a tumor that invades surrounding tissues, and is usually capable of producing metastases, may recur after attempted removal, and is likely to cause death of the host unless adequately treated) (see Steadman's Medical Dictionary, 26th Ed Williams & Wilkins, Baltimore, Md. (1995)).

The invention also provides a method of stabilizing or reversing a tumor load in a patient by administering to the patient an allogeneic tumor cell into which has been introduced a first nucleic acid encoding CD80 and a second nucleic acid encoding an HLA antigen.

In another embodiment, the invention provides a tumor cell, which can be a tumor cancer cell such as a lung cancer cell, genetically modified to express a nucleic acid encoding CD80 (B7.1) and a nucleic acid encoding an HLA antigen.

Exemplary HLA antigens include, but are not limited to, HLA A1, HLA A2, HLA A3, HLA A27, and the like. In a particular embodiment, the HLA antigen can be HLA A1 or HLA A2 (see Examples). One of skill in the art will appreciate that there are a number of different nucleic acid sequences encoding HLA antigens which may be used according to the invention without departing from the same (see below). Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of recombinant DNA technology include for example, Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); Kaufman et al., Eds., Handbook of Molecular and Cellular Methods in Biology in Medicine, CRC Press, Boca Raton (1995); McPherson, Ed., Directed Mutagenesis: A Practical Approach, IRL Press, Oxford (1991). Standard reference works setting forth the general principles of pharmacology include Goodman and Gilman's The Pharmacological Basis of Therapeutics, 10^{th} Ed., McGraw Hill Companies Inc., New York (2001). The compositions according to the invention are optionally formulated in a pharmaceutically acceptable vehicle with any of the well known pharmaceutically acceptable carriers, including diluents and excipients (see Remington's Pharmaceutical Sciences, 18th Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

In some embodiments, the cancer cell can be a lung tissue cancer cell (also referred to as “lung cancer cell”) such as an adenocarcinoma cell type, for example, the lung cancer cell can be the AD100 cell line, as exemplified hereinafter.

The invention additionally provides a method of stimulating an immune response to a tumor, for example, a cancer such as a lung cancer, in a patient by administering an allogeneic tumor cell genetically modified to express a nucleic acid encoding CD80 (B7.1) and a nucleic acid encoding an HLA antigen. The tumor cell can be a cancer cell, for example, a lung cancer tumor cell.

The methods of the present invention are intended for use with any subject that may experience the benefits of the methods of the invention. Thus, in accordance with the invention, “subjects”, “patients” as well as “individuals” (used interchangeably) include humans as well as non-human subjects, particularly domesticated animals.

In one embodiment, a method of the invention can include matching the HLA antigen to the individual administered the tumor lung cancer cell. Methods of determining HLA haplotypes are well known to those skilled in the art, for example, using well known serological assays using antibodies to HLA alleles or the mixed lymphocyte reaction. In a particular embodiment, a method of the invention can be performed with the HLA antigen HLA A1, HLA A2, HLA A3 or HLA A27. The methods of the invention can use various tumor cells (e.g., lung cancer cells) including, for example, an adenocarcinoma such as the AD100 cell line exemplified hereinafter.

In still another embodiment, the invention provides a method of inhibiting a tumor by administering an allogeneic tumor cell genetically modified to express a nucleic acid encoding CD80 (B7.1) and a nucleic acid encoding an HLA antigen. The tumor can be, for example, a cancer tumor cell such as a lung cancer tumor cell. In certain embodiments, the tumor inhibited is lung cancer by the administration of an allogeneic cancer cell modified to express CD80 (B7.1) and an HLA antigen.

As used herein, an “allogeneic cell” refers to a cell that is not derived from the individual to which the cell is to be administered, that is, has a different genetic constitution than the individual. An allogeneic cell is generally obtained from the same species as the individual to which the cell is to be administered. For example, the allogeneic cell can be a human cell, as disclosed herein, for administering to a human patient such as a cancer patient. As used herein, an “allogeneic tumor cell” refers to a tumor cell that is not derived from the individual to which the allogeneic cell is to be administered. Generally, the allogeneic tumor cell expresses one or more tumor antigens that can stimulate an immune response against a tumor in an individual to which the cell is to be administered. As used herein, an “allogeneic cancer cell,” for example, a lung cancer cell, refers to a cancer cell that is not derived from the individual to which the allogeneic cell is to be administered. Generally, the allogeneic cancer cell expresses one or more tumor antigens that can stimulate an immune response against a cancer in an individual to which the cell is to be administered, for example, a lung cancer.

As used herein, a “genetically modified cell” refers to a cell that has been genetically modified to express an exogenous nucleic acid, for example, by transfection or transduction. A cell can be genetically modified to express, for example, a nucleic acid encoding CD80 (B7.1) and/or a nucleic acid encoding an HLA antigen, as disclosed herein. When a cell is to be genetically modified to express more than one polypeptide, for example, CD80 (B7.1) and an HLA antigen, it is understood that the polypeptides can be encoded on separate nucleic acids (see Example 1) or on the same nucleic acid, if desired. Methods of genetically modifying a cell are well known to those skilled in the art.

The invention provides methods and compositions for stimulating an immune response in a cancer patient. The compositions and methods are particularly useful for stimulating an immune response against non-immunogenic tumors. As used herein, a non-immunogenic tumor is a tumor that does not elicit a spontaneous immune response detectable, for example, by appreciable stimulation of CD8 T cells that produce interferon-γ (IFNγ) in tumor infiltrating lymphocytes (TILs).

Traditionally, melanoma and other immunogenic tumors have been preferred for treatment by immunotherapy. In the present invention, non-immunogenic tumors are considered good targets for active immunotherapy because the tumor cells have not been immuno-selected for evasion of the CTL response. Exemplary non-immunogenic tumors include, but are not limited to, lung, pancreatic, and the like.

A particularly useful nonimmunogenic tumor type is non small cell lung cancer (NSCLC), as exemplified herein. NSCLC tumors are good targets for active immunotherapy, because they are non-immunogenic and do not spontaneously generate CTL responses. Therefore, NSCLC tumor cells have not developed evasive mechanisms towards cytotoxic T and natural killer (NK) cells, and NSCLC tumors are susceptible to cytotoxic attack. As disclosed herein, a composition of the invention was used to successfully slow tumor growth in NSCLC patients (see Examples II and III).

NSCLC tumors can also be genetically engineered to express and secrete gp96 and enhance the effectiveness of a vaccine because it combines adjuvant activity with polyvalent peptide specificity. Polyvalence prevents immunoselection and evasion. Tumor secreted gp96 activates dendritic cells (DC), natural killer cells (NK) and cytotoxic T lymphocytes (CTL), activating innate and adaptive immunity. Tumor cells can be killed by NK-specific mechanisms, by promiscuous killing of CD8 CTL through NKG2D, and by MHC restricted CD8 CTL activity. The activation of DC and NK by tumor secreted gp96 may also counteract the generation of immuno-suppressive CD4 regulatory cells found in NSCLC tumors. Tumor secreted gp96 stimulates antigen cross presentation via the CD91 receptor on DC and macrophages. NSCLC are known to share tumor antigens also found in melanoma and may be endowed with additional shared antigens. Therefore allogeneic, gp96 secreting tumor cells used as vaccine are expected to generate immunity to the patient's autologous tumor. Similarly, a composition of the invention containing an allogeneic tumor cell expressing CD80 and an HLA antigen can generate immunity to the patient's autologous tumor.

Lung tumors prevent priming of CTL by regulatory cells, by TGF-β secretion and by down regulation of MHC class I. Therefore, immunogenic vaccines are needed to generate a CTL response. Lung tumors are susceptible to CTL killing because they have not been selected for CTL evasion. Lung tumor TIL contain large numbers of CD4 regulatory cells suppressing priming. In contrast, melanoma TIL contain antigen specific CD8 CTL whose killing activity has been blocked, indicating that priming has taken place already. As disclosed herein, lung cancer patients were successfully treated with a vaccine containing an allogeneic tumor cell genetically modified to express CD80 (B7.1) and an HLA antigen (Examples II and III). Thus, immunotherapy (vaccine therapy) of NSCLC is useful for treating this otherwise deadly disease.

As disclosed herein, an adenocarcinoma is an exemplary lung cancer that can be used in compositions and methods of the invention to express CD80 (B7.1) and an HLA antigen. Other types of lung cancer are well known, and cells derived from other types of lung cancers can be similarly used in compositions and methods of the invention. Exemplary lung cancers include, for example, non-small cell lung cancer, which can be adenocarcinoma, squamous cell carcinoma, or large cell carcinoma, small cell lung cancer, and carcinoids. One skilled in the art can readily obtain tissue samples from various types of lung cancers and generate a cell line useful for treating a lung cancer, using methods similar to those disclosed herein. Similarly, other types of nonimmunogenic tumors can be used to generate allogeneic tumor cells that can be genetically modified to express CD80 (B7.1) and an HLA antigen and used to treat a similar type of tumor or a tumor expressing similar types of tumor antigens.

An exemplary allogeneic tumor cell is the AD100 cell line, which is a human lung adenocarcinoma cell line, as disclosed herein. Other lung cancer cell lines are well known to those skilled in the art and can be similarly used to generate an allogeneic cell genetically modified with CD80 (B7.1) and ann HLA antigen. For example, numerous cell lines, including lung cancer cell lines are well known and available from the American Type Culture Collection (ATCC; Manassas Va.). Exemplary NSCLC cell lines include, but are not limited to, NCI-H2126 [H2126] (ATCC CCL-256); NCI-H23 [H23] (ATCC CRL-5800); NCI-H1299 [H1299] (ATCC CRL-5803); NCI-H358 [H358] (ATCC CRL-5807); NCI-H810 [H810] (ATCC CRL-5816); NCI-H522 [H522] (ATCC CRL-5810); NCI-H1155 [H1155] (ATCC CRL-5818); NCI-H647 [H647] (ATCC CRL-5834); NCI-H650 [H650] (ATCC CRL-5835); NCI-H838 [H838] (ATCC CRL-5844); NCI-H920 [H920] (ATCC CRL-5850); NCI-H969 [H969] (ATCC CRL-5852); NCI-H1385 [H1385] (ATCC CRL-5867); NCI-H1435 [H1435] (ATCC CRL-5870); NCI-H1437 [H1437] (ATCC CRL-5872); NCI-H1563 [H1563] (ATCC CRL-5875); NCI-H1568 [H1568] (ATCC CRL-5876); NCI-H1581 [H1581] (ATCC CRL-5878); NCI-H1623 [H1623] (ATCC CRL-5881); NCI-H1651 [H1651] (ATCC CRL-5884); NCI-H1693 [H1693] (ATCC CRL-5887); NCI-H1703 [H1703] (ATCC CRL-5889); NCI-H1734 [H1734] (ATCC CRL-5891); NCI-H1755 [H1755] (ATCC CRL-5892); NCI-H1770 [H1770] (ATCC CRL-5893); NCI-H1793 [H1793] (ATCC CRL-5896); NCI-H1838 [H1838] (ATCC CRL-5899); NCI-H1869 [H1869] (ATCC CRL-5900); NCI-H1915 [H1915] (ATCC CRL-5904); NCI-H1944 [H1944] (ATCC CRL-5907); NCI-H1975 [H1975] (ATCC CRL-5908); NCI-H1993 [H1993] (ATCC CRL-5909); NCI-H2023 [H2023] (ATCC CRL-5912); NCI-H2030 [H2030] (ATCC CRL-5914); NCI-H2073 [H2073] (ATCC CRL-5918); NCI-H2085 [H2085] (ATCC CRL-5921); NCI-H2087 [H2087] (ATCC CRL-5922); NCI-H2106 [H2106] (ATCC CRL-5923); NCI-H2110 [H2110] (ATCC CRL-5924); NCI-H2135 [H2135] (ATCC CRL-5926); NCI-H2172 [H2172] (ATCC CRL-5930); NCI-H2228 [H2228] (ATCC CRL-5935); NCI-H2291 [H2291] (ATCC CRL-5939); NCI-H2342 [H2342] (ATCC CRL-5941); NCI-H2347 [H2347] (ATCC CRL-5942); NCI-H2405 [H2405] (ATCC CRL-5944); NCI-H2444 [H2444] (ATCC CRL-5945); and NCI-H2122 [H2122] (ATCC CRL-5985). These and other tumor cell lines, particularly those of nonimmunogenic tumors, can similarly be used in compositions and methods of the invention.

As disclosed herein, these and other tumor cell lines can be genetically modified to express exogenous molecules that enhance an immune response to tumor antigens. Such molecules include, but are not limited to, CD80 (B7.1), human HLA antigens, for example, HLA A1, A2, A3, A27, and the like. One skilled in the art can readily obtain appropriate sequences encoding such molecules using well known methods. One skilled in the art will readily understand that variants of such molecules are available or can be readily obtained using well known methods. Based on known complete or partial sequences, one skilled in the art can use well known molecular biology methods to obtain nucleic acid sequences suitable to genetically modify a tumor cell, as disclosed herein. It is understood that these exemplary sequences as well as natural variations of such sequences are considered within the scope of the invention.

Exemplary nucleic acid sequences encoding molecules that enhance an immune response are available, for example, from GenBank, including complete and partial cDNA sequences as well as genomic sequences, and such sequences can be used to obtain nucleic suitable nucleic acid sequences encoding desired immune enhancing molecules. A representative selection of such sequences available from GenBank include, but are not limited to, GenBank accession numbers NT_{—}005612; NM_{—}012092; NM_{—}175862; NM_{—}006889; NM_{—}005191; BC042665; NM_{—}012092; NM_{—}175862; NM_{—}006889; NM_{—}152854; NM_{—}005214; NM_{—}005514; NM_{—}002116; Z70315; NM_{—}002127; AH013634; L34703; L34734; AF389378; U30904; AH006709; AH006661; AH006660; X55710; U04244; U35431; M24043; U03859; NM_{—}005514; NM_{—}002116; Z30341; NM_{—}012292; NM_{—}002127; NM_{—}002117; AH007560; AH000042; AB048347; AB032594; AJ293264; AJ293263; AB030575 AB030574; AB030573; AF221125; AF221124; AH009136; X60764; AB032597; L17005; Y13267; AH003586; Z46633; Z27120; Z33453; Z23071; X02457; X57954; K02883; U21053; U04243; U18930; L36318; L36591; L38504; L33922; M20179; M20139; M24042; M15497; M31944; U04787; U01848; M27537; U11267; U03907; U03863; U03862; U03861; NM_{—}002116; L34724; L34723; L34721; L34737; L34701; Z97370; L15370; AH003070; M20179; M16273; M16272; M15497; M19756; M19757; NT_{—}008413, and the like.

The compositions and methods of the invention are useful for stimulating an immune response against a tumor. Such immune response is useful in treating or alleviating a sign or symptom associated with the tumor. Such an immune response can ameliorate a sign or symptom associated with a lung cancer. As used herein, by “treating” is meant reducing, preventing, and/or reversing the symptoms in the individual to which a compound of the invention has been administered, as compared to the symptoms of an individual not being treated according to the invention. A practitioner will appreciate that the compositions and methods described herein are to be used in concomitance with continuous clinical evaluations by a skilled practitioner (physician or veterinarian) to determine subsequent therapy. Hence, following treatment the practitioners will evaluate any improvement in the treatment of the pulmonary inflammation according to standard methodologies. Such evaluation will aid and inform in evaluating whether to increase, reduce or continue a particular treatment dose, mode of administration, etc.

The methods of the invention can thus be used to treat a tumor, including, for example, a cancer such as a lung cancer. The methods of the invention can be used, for example, to inhibit the growth of a tumor by preventing further tumor growth, by slowing tumor growth, or by causing tumor regression. Thus, the methods of the invention can be used, for example, to treat a cancer such as a lung cancer. It will be understood that the subject to which a compound of the invention is administered need not suffer from a specific traumatic state. Indeed, the compounds of the invention may be administered prophylactically, prior to any development of symptoms (e.g., a patient in remission from cancer). The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a therapeutically effective amount of a composition of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the composition of the invention may be lowered or increased by fine tuning and/or by administering more than one composition of the invention (e.g., by the concomitant administration of two different genetically modified tumor cells), or by administering a composition of the invention with another compound to enhance the therapeutic effect (e.g., synergistically). The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

The methods of the invention can thus be used, alone or in combination with other well known tumor therapies, to treat a patient having a tumor. One skilled in the art will readily understand advantageous uses of the invention, for example, in prolonging the life expectancy of a lung cancer patient and/or improving the quality of life of a lung cancer patient.

Current recommendations for NSCLC patients with locally-advanced inoperable disease (stage IIIB) include platinum-based chemotherapy plus radiation therapy, and chemotherapy alone for patients with metastases (stage IV) (Clinical practice guidelines for the treatment of unresectable non-small-cell lung cancer; Adopted on May 16, 1997 by the American Society of Clinical Oncology, J. Clin. Oncol. 15: 2996-3018, 1997). Results of these approaches are nevertheless poor, and the increase in survival is limited. The largest meta-analysis published to date concluded that chemotherapy increases the chance of 1-year survival by 10% and median survival by 6 weeks (Chemotherapy in non-small cell lung cancer: A meta-analysis using updated data on individual patients from 52 randomizsed clinical trials. Non-Small Cell Lung Cancer Collaborative Group. BMJ 311:899, 1995). A recent report from the Big Lung Trial group (BLT) reported similar results (Stephens et al., Proc. Am. Soc. Clin. Oncol. 21:2002 (abstract 1661)). In phase III clinical trials, patients with metastatic disease have a median survival of less than 1 year (Schiller, et al., N. Engl. J. Med. 346:92-98 (2002)). Two phase III trials showed that after failure of first-line chemotherapy, only 6% of patients receiving standard second-line chemotherapy could expect to respond, with median survival being approximately 6 months (Shepherd, et al., J. Clin. Oncol. 18:2095-2103 (2000); Fossella, et al., J. Clin. Oncol. 18:2354-2362 (2000)). In the experiments described herein, the group of patients had a very poor prognosis as a result of their relapsed or metastatic disease status, and most patients had been unsuccessfully treated with surgery, radiation, and/or palliative chemotherapy, resulting in a projected survival of less than 6 months.

A vaccination approach such as that disclosed herein can be an effective means of inducing immune response in patients with nonimmunogenic tumors. There is evidence that NSCLC tumors contain tumor antigens (Yamazaki, et al., Cancer Res. 59:4642-4650 (1999); Weynants, et al., Am. J. Respir. Crit. Care Med. 159:55-62 (1999); Bixby, et al., Int. J. Cancer 78:685-694 (1998); Yamada, et al., Cancer Res. 63:2829-2835 (2003)). However, it has been thought that lung tumors are poor candidates for immunotherapy because they are poorly immunogenic and are potentially immunosuppressive (Woo, et al., J. Immunol. 168:4272-4276 (2002); Woo et al., Cancer Res. 61:4766-4772 (2001); Neuner, et al., Int. J. Cancer. 101:287-292 (2002); Neuner, et al., Lung Cancer 34 (supplement 2):S79-82 (2001); Dohadwala, et al., J. Biol Chem. 276:20809-20812 (2001)), thereby anergizing or tolerizing T-cells (Schwartz, J. Exp. Med. 184:1-8 (1996); Lombardi, et al., Science 264:1587-1589 (1994)). Lung tumors, therefore, have not been subjected to immune attack, and hence have not been able to evolve evasive mechanisms to resist immune effector cells. Lung tumors, unlike immunogenic tumors that harbor tumor-infiltrating lymphocytes, thus may succumb to killer CTLs, especially in light of the involvement of CD8 CTLs in tumor rejection in a number of model systems (Podack, J. Leukoc. Biol. 57:548-552 (1995)).

As disclosed herein, an allogeneic whole cell vaccine was chosen because whole cell vaccines have given the best clinical results so far. For example, statistically significant survival benefit occurred when a whole cell melanoma vaccine was administered (Morton, et al., Ann. Surg. 236:438-449 (2002)). In contrast, vaccine directed at a single epitope may have limited utility due to tumor escape mutants (Velders, et al., Semin. Oncol. 25:697-706 (1998)). The additional advantage of a whole cell vaccine approach is that it does not require a priori delineation of specific lung tumor antigens. If vaccination is successful and CTLs are generated, as was found in the experiments disclosed herein, the responsible antigenic sites can be identified later. Allogeneic cell-based vaccines offer a good alternative to autologous vaccines under the assumption that lung tumor antigens are shared in lung tumors of different patients, and the antigens can be cross-presented by the patients' antigen-presenting cells. Although there is only limited evidence for shared antigens in lung tumors (Yamazaki, et al., Cancer Res. 59:4642-4650 (1999); Yamada, et al., Cancer Res. 63:2829-2835 (2003)), this has been shown in other tumors (Fong, et al., Annu. Rev. Immunol. 18: 245-273 (2000); Boon, et al., Annu. Rev. Immunol. 12:337-365 (1994)).

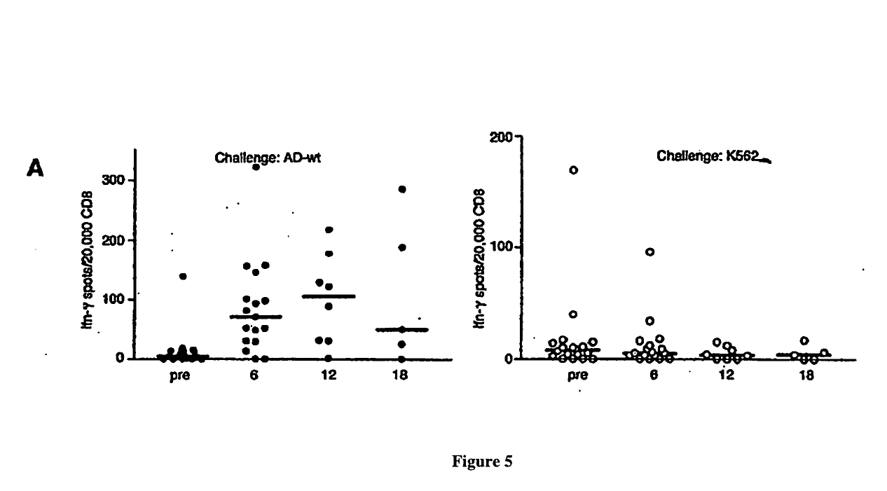

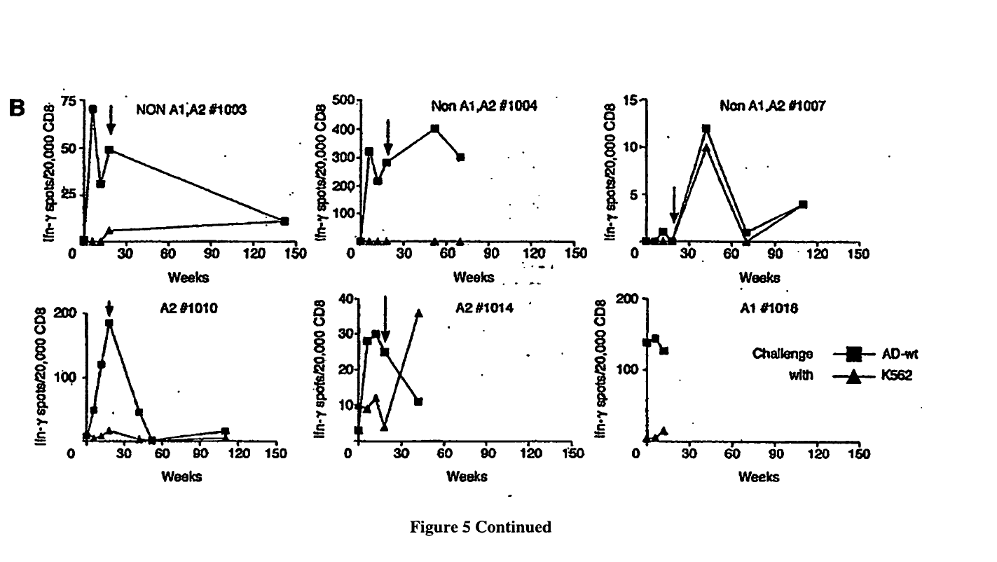

To obtain direct evidence that the CD8 cells generated in response to allogeneic vaccination recognize autologous tumor cells, tumor specimens should be obtained at the time of surgery. Tumor specimens were not available in the trial of patients disclosed herein with advanced disease (see Examples II and III). However, the prolonged maintenance of a high frequency of patient CD8 cells reacting to AD100 in vitro, and their increase in some patients (No. 1004 and No. 1007; FIG. 5) even after cessation of external vaccination, is consistent with the immune stimulation of patient CD8 cells by the autologous tumor and their cross-reaction with the allogeneic vaccine.

In the experiments disclosed herein, although only one patient had a partial response, five other patients had stable disease. Enhanced immune reactivity was demonstrated by a CD8-mediated tumor-specific immune response. The fact that six (32%) of 19 patients with very poor prognosis exhibited disease stabilization of a rapidly lethal condition, with median survival of the whole cohort reaching 18 months despite far-advanced disease, is encouraging. The results disclosed herein indicate that tumor progression is slowed by vaccination, and that this effect occurs regardless of whether or not patients are allogeneic to the HLA A1 or A2 locus of the vaccine. The findings also indicate that indirect antigen presentation can be effective in promoting antitumor activity and that allogeneic MHC molecules enhance the effect.

In the results disclosed herein, the vaccine was well tolerated and the patients' quality of life was very good, thus improving patient outcome. Because this is an immunologic product, it was assumed that some immune-mediated side effects would be anticipated. Probable examples of such phenomena of expected tolerable side effects were, for example, the local erythema at the vaccination site in five patients, and the episode of arthritic pain experienced by one patient (see Example III).

A composition of the invention containing a tumor cell genetically modified to express CD80 and an HLA antigen can be combined with a physiologically acceptable carrier useful in a vaccine by including any of the well known components useful for immunization. The components of the physiological carrier are intended to facilitate or enhance an immune response to an antigen administered in a vaccine. The formulations can contain buffers to maintain a preferred pH range, salts or other components that present the antigen to an individual in a composition that stimulates an immune response to the antigen. The physiologically acceptable carrier can also contain one or more adjuvants that enhance the immune response to the antigen. Formulations can be administered subcutaneously, intramuscularly, intradermally, or in any manner acceptable for immunization.

An adjuvant refers to a substance which, when added to an immunogenic agent of the invention such as tumor cell genetically modified to express CD80 and an HLA antigen, nonspecifically enhances or potentiates an immune response to the agent in the recipient host upon exposure to the mixture. Adjuvants can include, for example, oil-in-water emulsions, water-in oil emulsions, alum (aluminum salts), liposomes and microparticles, such as, polysytrene, starch, polyphosphazene and polylactide/polyglycosides. Adjuvants can also include, for example, squalene mixtures (SAF-I), muramyl peptide, saponin derivatives, mycobacterium cell wall preparations, monophosphoryl lipid A, mycolic acid derivatives, nonionic block copolymer surfactants, Quil A, cholera toxin B subunit, polyphosphazene and derivatives, and immunostimulating complexes (ISCOMs) such as those described by Takahashi et al. Nature 344:873-875 (1990). For veterinary use and for production of antibodies in animals, mitogenic components of Freund's adjuvant (both complete and incomplete) can be used. In humans, Incomplete Freund's Adjuvant (IFA) is a useful adjuvant. Various appropriate adjuvants are well known in the art (see, for example, Warren and Chedid, CRC Critical Reviews in Immunology 8:83 (1988); Allison and Byars, in Vaccines: New Approaches to Immunological Problems, Ellis, ed., Butterworth-Heinemann, Boston (1992)). Additional adjuvants include, for example, bacille Calmett-Guérin (BCG), DETOX (containing cell wall skeleton of Mycobacterium phlei (CWS) and monophosphoryl lipid A from Salmonella minnesota (MPL)), and the like (see, for example, Hoover et al., J. Clin. Oncol., 11:390 (1993); Woodlock et al., J. Immunotherapy 22:251-259 (1999)).

The compositions and methods of the invention disclosed herein are useful for treating a patient having a tumor. Although particular embodiments are exemplified with lung cancers, it is understood that a similar approach can also be used to treat other types of tumors, including cancers, using suitable allogeneic cells.

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims

### EXAMPLE I

### Allogeneic Vaccination with a B7.1 HLA-A Gene-modified Adenocarcinoma Cell Line in Patients with Advanced Non-Small-Cell Lung Cancer

This example describes the protocol used for allogeneic vaccination with a B7.1 HLA-A gene-modified adenocarcinoma cell line in patients with advanced non-small-cell lung cancer (NSCLC). This example describes the experimental protocol used.

The following experiments were designed (a) to measure whether CD80 and HLA A transfected, allogeneic lung tumor cells used for immunotherapy can elicit tumor specific CD8-CTL activation and expansion, assessed by ELIspot for IFN-γ; (b) to evaluate the safety and toxicity of administering allogeneic tumor cell vaccines transfected with B7.1 and HLA A1 or A2 in patients with Non-Small Cell Lung Carcinoma (NSCLC); and (c) to evaluate the antitumor effect of this B7.1 vaccine in clinical outcomes for patients with NSCLC.

Selection of Patients. Initially, fifteen patients with newly diagnosed or relapsed metastatic non-small cell lung cancer (NSCLC) were treated. The analysis of these 15 patients is described in Example II. An additional four patients were added, for a total of 19 patients, and the further results with the 19 patients are described in Example III. The patients had already failed chemotherapy, radiotherapy, surgery or a combination of all. Eligibility criteria were as follows: age>18 years, Eastern Cooperative Oncology Group (ECOG) performance status 0-2, measurable disease, signed informed consent, and histologically confirmed NSCLC (stage IIIB with malignant pleural effusion, stage IV, or recurrent). Patients with brain metastasis were included if these were already treated. Patients were not eligible for study if they were receiving chemotherapy, radiation therapy or a biologic modifying agent or during the preceding 4 weeks. All patients were treated in the outpatient clinic at Sylvester Comprehensive Cancer Center/University of Miami. A complete history and physical exam was performed, including weight and vital signs, with performance status assessed by ECOG criteria. The following tests were performed prior to enrollment: complete blood count; platelet count; chemistries (uric acid, calcium, phosphorus, transaminases including serum glutamic-oxaloacetic transaminase (SGOT) and serum glutamic-pyruvic transaminase (SGPT), alkaline phosphatase, lactate dehydrogenase (LDH), total and direct bilirubin, blood urea nitrogen (BUN), creatinine, albumin, total protein, electrolytes, and glucose); and electrocardiogram (EKG). HLA typing was obtained. Patients were followed twice monthly while being vaccinated, with tumor response assessed by computed tomography (CT) scans. Tumor measurements were obtained from the results of radiographic studies, including CT scans of relevant sites.

Vaccine Cell Line and Genetic Modification. A human lung adenocarcinoma cell line was established in 1994 by Dr. N. Savaraj (University of Miami, Department of Medicine) from a biopsy of a lung cancer patient, designated as AD100. The patient was a 74 year old white male who presented in 1993 with initial symptoms of pelvic pain from bone erosion of the iliac crest due to metastatic pulmonary adenocarcinoma. Cancer cells for culture were obtained by bone marrow aspiration from the area of pelvic bone destruction. The patient was treated with radiation therapy to the pelvis, but expired one month after diagnosis. The cell line derived from this patient has been kept in culture in standard medium (described below) and is free of contamination by mycoplasma, virus or other adventitious agents. The cell line is homogeneous, adherent to plastic, and grows with a rate of division of approximately 26 h.

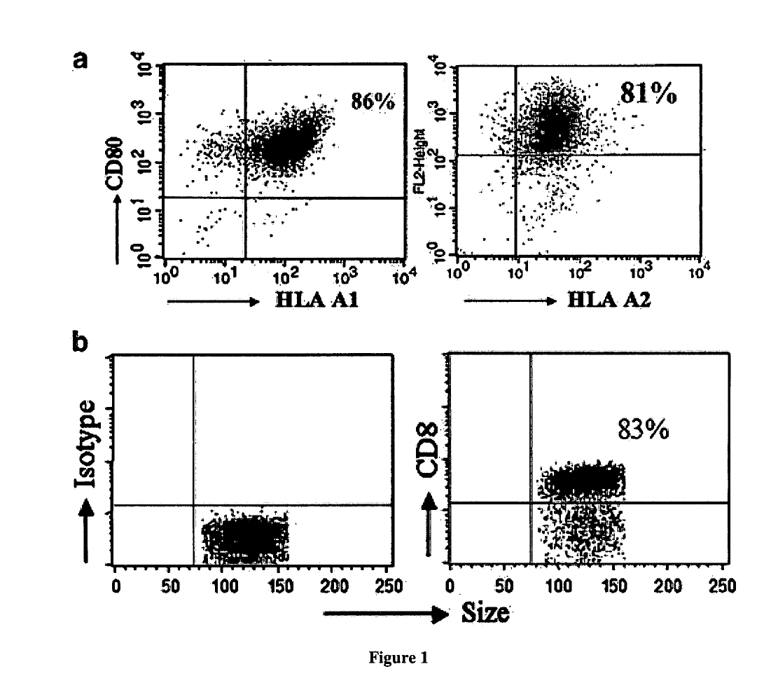

Genetic Modification. AD100 was transfected with plasmid cDNA, pBMG-Neo-B7.1 and pBMG-His-HLA A2 or with B45-Neo-CM-A1-B7.1 (Yamazaki et al., Cancer Res., 59:4642, 1999) Transfected cells were selected with G418 and histidinol. Verification of correct sequences was based on restriction analysis and the expression of the relevant gene products, namely G418 or histidinol resistance for the vector sequence, HLA A1, A2, and B7.1 expression for the transfected cDNA. The cells were irradiated to prevent their replication, for example, with 12,000 Rads in a cobalt (Co) irradiator, and stored frozen in 10% DMSO in aliquots of 5×10⁷ cells until use. Upon replating in tissue culture the cells appeared viable for about 14 days but were unable to form colonies, indicating their inability to replicate. They were therefore considered safe for use as vaccine cells. The minimum requirement for their use as vaccine was the co expression of HLA A1 or A2 plus B7.1 on at least 70% of the cells, as shown in FIG. 1A for representative batches of vaccine cells. The untransfected AD100 line was negative by FACS for staining with anti HLA A1 or A2 or B7.1. FIG. 1A shows the quality control by flow cytometric analysis of CD80 and HLA A1 or A2 transfected AD100 vaccine cells used for immunization.

Immunizations. Intracutaneous injections were given at multiple body sites to reduce the extent of local skin reactions. Patients who were HLA A1 or A2 received the corresponding HLA-matched vaccine, whereas patients who were neither HLA A1 nor HLA A2 received HLA A1-transfected vaccine (that is, HLA-unmatched vaccine). On a given vaccination day, the patient received the total dose of 5×10⁷ irradiated cells (12,000 rad) divided into two to five aliquots for administration as two to five intradermal injections of each aliquot in an extremity, spaced at least 5 cm at needle entry from the nearest neighboring injection. A total of nine immunizations (4.5×10⁸ cells) were given over the course of therapy, one every two weeks, provided that no tumor progression occurred under therapy (Table 1). On subsequent vaccinations, the injection sites were rotated to different limbs in a clockwise manner. One course of vaccination comprised three biweekly injections. Patients with evidence of stable disease or responding NSCLC by imaging evaluation (CT Scans) and none to moderate toxicity (grade≦2) were treated with an additional course at the same dose. The second course of injections started two weeks after the third vaccination that completed the first course. In the absence of tumor progression by CT scans and with no severe or life-threatening toxicity (grade≧3), a third course at the same dose of therapy was given, starting two weeks after the third vaccination of the second course of therapy. Clinical, toxicity, and immunologic evaluations by blood tests prior to and after each course were performed was done. Patients were followed clinically weekly during the study, including monitoring blood counts and basic chemistries (Table 1).

Table 1 shows the treatment and evaluation schedule of NSCLC (IIIB/IV) patients. Patients were immunized nine times in biweekly intervals, as discussed above. Immunological assays were done prior to and after each of three immunizations.

TABLE 1
Immunizations and Immunological Evaluations

| | Study | | | | | | | | | | | | |
|---|---|---|---|---|---|---|---|---|---|---|---|---|---|
| | Entry | Course 1 | | | | Course 2 | | | | Course 3 | | | |
| Weeks on Study | 1 | 2 | 4 | 6 | 7 | 8 | 10 | 12 | 13 | 14 | 16 | 18 | 19 |
| Pre-Entry Evaluation | x | | | | | | | | | | | | |
| Immunization # | | 1 | 2 | 3 | | 4 | 5 | 6 | | 7 | 8 | 9 | |
| Clinical Evaluation | x | x | x | x | x | x | x | x | x | x | x | x | x |
| Toxicity Evaluation | | x | x | x | x | x | x | x | x | x | x | x | x |
| Immunological Evaluation # | 1 | | | | 2 | | | | 3 | | | | 4 |

Immunological Testing. Immunological tests were performed included skin tests delayed-type hypersenstivity (DTH) and enzyme-linked immunospot (ELISPOT) assays for interforn-γ IFN-γ. Immune responses mediated by CD4 cells were examined by DTH-reaction following intradermal injection of 10⁵ A1, A2 or untransfected AD100-B7 vaccine cells. Purified CD8 cells were obtained from patients prior to and after each course of three immunizations. CD8 cells were enriched by negative depletion with anti-CD56, anti-CD4 and other antibodies using the Spin-sep prep (Stem Cell Technologies; Vancouver, Canada). Purity was better than 80% (FIG. 1B) the primary contaminating cells being B cells (not shown). CD8 cells were frozen in 10% dimethylsulfoxide (DMSO) and 20% fetal calf serum (FCS) containing medium for analysis until all vaccinations of a study patient were completed. Analysis for pre-immune and post-vaccination ELISPOT frequency was carried out on the same day in the same micro titer plate. Assays were done in quadruplicate, stimulating 2×10⁴ purified patient CD8 cells with, respectively, 10³ A1 or A2 transfected or untransfected AD100, with K562 or with media only for three days and determining the frequency of IFN-γ producing cells by ELISPOT. Immune assays were performed prior to immunization and after 3, 6, and 9 immunizations.

Statistical Analysis. Patient characteristics are presented as counts with percentages, or as mean values and range. Overall survival, estimated by the Kaplan-Meier product-limit method, is defined as time from enrollment onto study until death from any cause. In the absence of death, follow-up was censored at the date of last patient contact. Univariate and multivariate proportional hazards regression were used to determine whether patients' survival time was related to age (continuous), sex, race (other versus white non-Hispanic), tumor pathology (adenocarcinoma versus other), and HLA-matching of vaccine. Logistic regression was used for the corresponding analyses of clinical response. For hazard ratios and the percentage of patients surviving, 90% confidence intervals (CIs) L₉₀-U₉₀ are reported. These can be interpreted as providing 95% confidence that the parameter being estimated, such as the hazard ratio, exceeds L₉₀.

### EXAMPLE II

### Specific CD8 T Cell Response of Advanced Lung Cancer Patients to Whole Cell Immunization with an Allogeneic Vaccine

This example describes the results of a 15 patient group study on whole cell immunization with an allogeneic vaccine.

Patients with advanced NSCLC stage IIIB/IV were HLA typed. HLA A1 positive patients received the AD-A1-B7 vaccine; HLA A2 positive patients received the AD-A2-B7 vaccine; and patients that were neither HLA A1 nor A2 positive received either the AD-A1-B7 or AD-A2-B7 vaccine. The frequency of IFN-γ secreting CD8 cells was determined by ELISPOT after restimulation of purified patient-CD8 cells in vitro with HLA A1 or A2 transfected or untransfected AD100. Controls included stimulation with K562 and incubation of CD8 cells without stimulator cells.

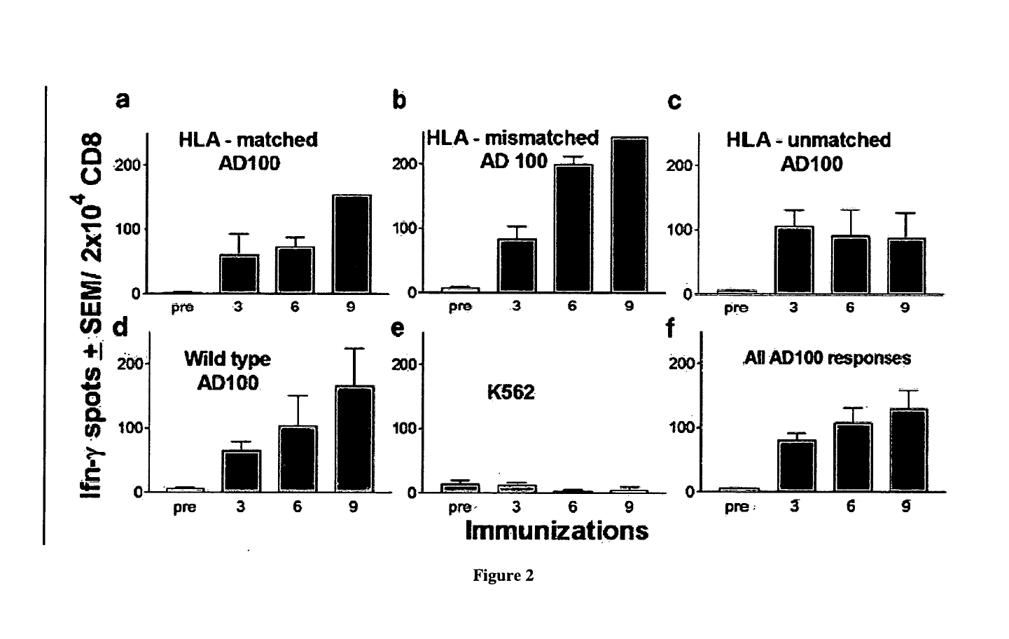

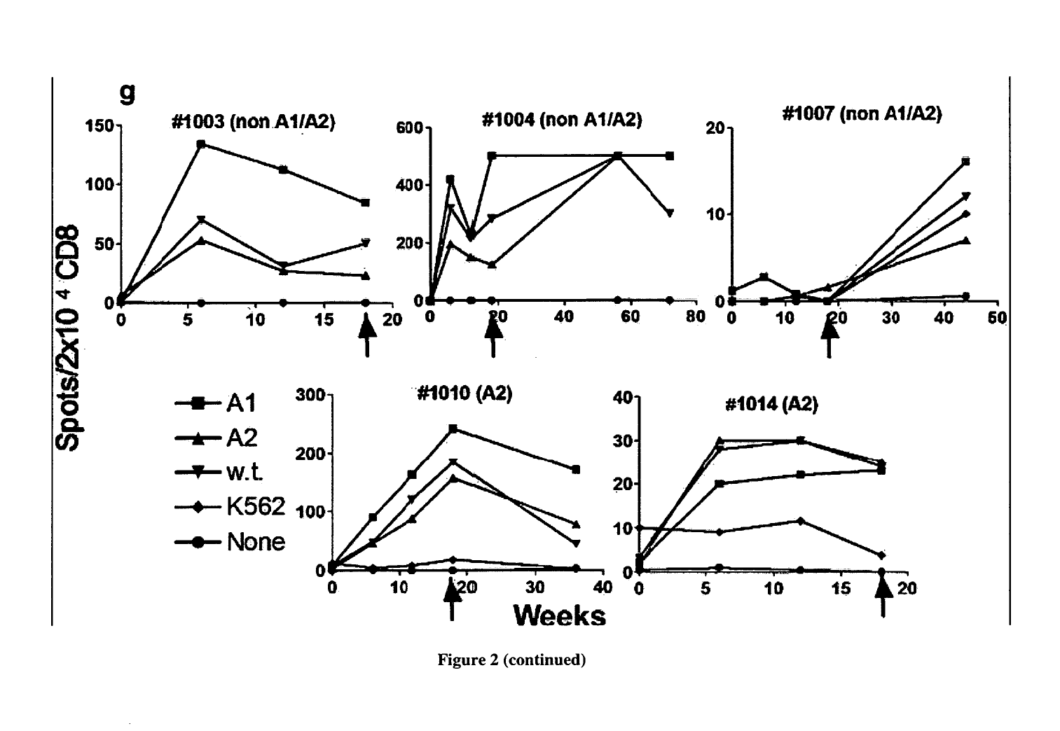

ELISPOT responses of immunized tumor patients are presented as HLA matched responses (FIG. 2A), representing the number of IFN-γ secreting CD8 cells obtained from HLA A1 or A2 patients challenged in vitro for three days with HLA A1 or A2 transfected AD100 cells, respectively. HLA mismatched responses indicate the number of spots formed when CD8 cells from A1 or A2 patients were challenged with A2 or A1 transfected AD100, respectively (FIG. 2B). The matched response increased 15-fold, from 6±4 (standard error of the mean, SEM) IFN-γ secreting, pre-immune CD8 cells (per 20 thousand) to maximal 90±35 (SEM) IFN-γ secreting cells after six immunizations and remained at this level during the next three immunizations. The mismatched response increased 5.7 fold, from 24±18 to 142±42 maximal. Included in this group of nine patients is the one patient who showed no response (0 spots) before or after three immunizations, at which time the tumor progressed and the patient was taken off trial.

The remaining 5 patients were negative for HLA A1 or A2. These patients CD8 response to challenge with A1 or A2 transfected AD100 is shown as unmatched response in FIG. 2C. The frequency of IFN-γ secreting CD8 cells increased 21-fold from 4.8±1.8 pre-immune to 105±24 after three immunizations and stayed constant throughout the trial. This increase in frequency is similar to that of all patients' CD8 cells when challenged with the untransfected wild type AD100 (FIG. 2D). Finally, the specificity of the response is evident from the absence of an increase of the response to K562 (FIG. 2E) or of unchallenged CD8 cells. The CD8 response to K562 and to AD100 in its w.t. form or after genetic modification is significantly different at each time point after vaccination (FIG. 2F).

The CD8 response listed in Table 2 reports the response to the matched vaccine for A1 or A2 positive patients. For non A1, A2 patients, it is the response to AD100-A2. One of 15 patients could not be analyzed due to renal failure unrelated to the trial prior to completing the first course of immunization. Of the fifteen patients treated, five patients had clinical responses: one partial response (PR), and four patients with stable disease (SD). Four of these patients with clinical responses, (PR+3 SD), are still alive with stabilization of their diseases without further therapy for: 31, 28, 25, and 12 months. The patient that died, originally had SD for 5 months then progressed and died 15 months later in spite of several courses of palliative chemotherapy. In contrast, nine of the other ten patients that did not respond to the vaccination are deceased except one patient who achieved stable disease after therapy with Iressa™. Table 2 summarizes the data for all patients, including pre-trial treatment, clinical response to immunization and immune response. Patients that had progressive disease while under treatment went off study as indicated in Table 2.

Table 2 shows a summary of clinical responses, immunological CD8 responses, survival and pretreatment of fifteen patients with advanced stage IIIB/IV NSCLC treated with allogeneic B7/HLA A transfected NSCLC vaccine. The abbreviations in Table 2 are: PD—progressive disease; NE—not evaluable for immune response, but included in survival analysis on the right; PR—partial response; SD—sable disease; C—chemotherapy; R—radiation; S—surgery. Survival indicates time of survival since study entry; + indicates patient alive; n.d. no done, patients off study because of progression.

TABLE 2
Summary of Clinical Responses, Immunological CD8 Responses,
Survival and Pretreatment of Fifteen NSCLC Patients.

| | | | | | | Ifn-γ producing CD8 cells to AD100-HLA | | | |
|---|---|---|---|---|---|---|---|---|---|
| | | Fold | | | Time to | challenge (spots per 20,000) | | | |
| Patient # | | Titer | Previous | Survival | Progression | Pre- | 1st | 2nd | 3rd |
| HLA | Response | increase | TX | (mos) | (mos) | immune | course | course | course |
| 1005 A1 | PD | 190 | C + R | 10 | — | 0 | 190 | n.d. | n.d. |
| 1012 A1 | NE | NE | C | 15 | — | 0.2 | n.d. | n.d. | n.d. |
| 1001 A2 | PD | 25 | C + S | 18 | — | 0 | 25 | n.d. | n.d. |
| 1002 A2 | PD | 1.6 | C + S | 22 | — | 41 | 65 | n.d. | n.d. |
| 1009 A2 | PD | 6.5 | C | 3 | — | 2 | 13 | n.d. | n.d. |
| 1010 A2 | PR | 41 | S | 27+ | 3 | 3.8 | 46 | 88 | 157 |
| 1011 A2 | PD | 19 | C | 11 | — | 3 | 30 | 57 | n.d. |
| 1013 A2 | PD | 34 | C + R + S | 2 | — | 5.2 | 164 | 178 | n.d. |
| 1014 A2 | SD | 19 | C + S | 13+ | 3 | 1.6 | 30 | 30 | 25 |
| 1015 A2 | PD | 0 | C + R | 7 | — | 0 | 0 | nd | nd |
| 1003 non | SD | 134 | S | 31+ | 26+ | 1 | 134 | 113 | 84 |
| 1004 non | SD | 424 | C + R | 23 | 11 | 0 | 424 | 232 | >450 |
| 1006 non | PD | 9.3 | C + S | 30+ | — | 16 | 150 | n.d. | n.d. |
| 1007 non | SD | 14 | C + R + S | 29+ | 23+ | 1.2 | 2.8 | .8 | 0/17 |
| 1008 non | PD | 32 | C | 6 | — | 5.6 | 178 | n.d. | n.d. |

Five patients had a clinical response and the frequency of IFN-spot forming CD8 cells increased upon successive immunization as measured by challenge ex vivo with transfected or untransfected AD100, while the reactivity to K562 remained low and unchanged (FIG. 2E). In three of the clinically responding patients (FIG. 2; 1004, 1007, 1010), blood samples were obtained after completion of the 18 week treatment period at 35 to 75 weeks post trial entry and showed still a considerable titer of CD8 cells responding to AD100 (FIG. 2G). Indeed, in two of two patients (1004, 1007), the titer increased further even after immunization was ended at 18 weeks.

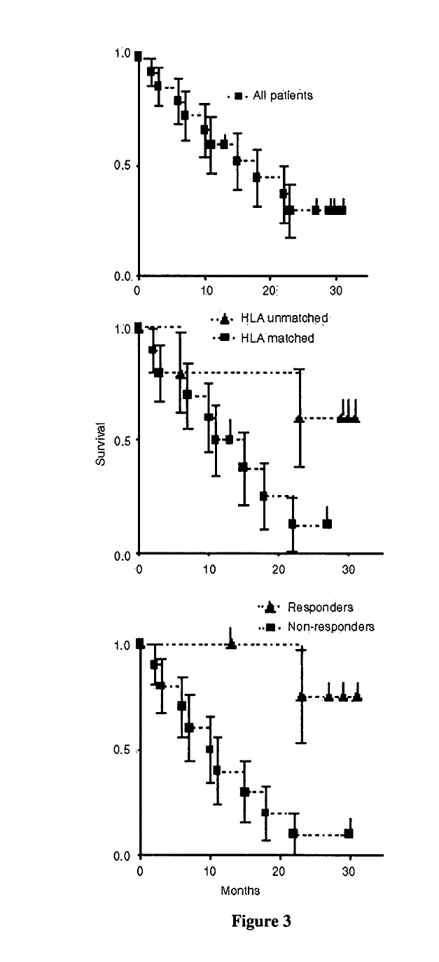

The median survival time of all patients at the time of analysis was 18 months, exceeding the expected median survival time of less than one year for this group of patients (FIG. 3). 90% confidence intervals are shown in FIG. 3. Analysis of survival by MHC matching and by clinical response revealed that HLA unmatched patients showed a survival advantage that with p=0.07 was not statistically significant while clinical responders had a significant (p=0.008) survival advantage when compared to non responders.

Safety. None of the 15 patients entered into the trial experienced any treatment related serious adverse events, defined as deaths or events requiring hospitalization. Treatment related side effects consisted of local erythema and swelling that resolved in three to four days. One patient complained about transient arthralgias that may have been treatment related. One patient died within 30 days of the last immunization due to pulmonary failure; one patient who had previous episodes of pericarditis experienced pericardial effusion during the last course of immunization, requiring a pericardial window. No tumor cells were detected in the fluid; the patient responded to immunization and is still in stable disease. As mentioned above, one patient had renal failure prior to completion of one course of immunization. None of these events were deemed likely to be treatment related by an independent safety monitoring board.

### EXAMPLE III

### Further Characterization of Advanced Lung Cancer Patients to Whole Cell Immunization with an Allogeneic Vaccine

This example describes a continuation of the study described in Example II, including additional patients and time of study.

Experiments were performed essentially as described in Example II and Raez et al., J. Clin. Oncol. 22:2800-2807 (2004).

Patient Characteristics. The characteristics of the 19 study patients are outlined in Table 3. Eastern Cooperative Oncology Group performance status was 0 to 1 in 18 patients (74%). Thirteen patients received vaccine matched for HLA, either A1 (three patients) or A2 (10 patients), whereas the six patients who were non-A1 and non-A2 received unmatched vaccine (that is, HLA-A1 vaccine). While HLA A matched patients may be able to mediate CD8 responses by direct antigen presentation by the vaccine cells, it was reasoned that unmatched patients may, nonetheless, mount a CD8 response via indirect antigen presentation after vaccine cell death and antigen uptake by antigen presenting cells. Before being enrolled on study, all patients had been previously treated: nine (47%) with surgery, six (32%) with radiation therapy, and 17 (89%) with chemotherapy. Among the chemotherapy-treated patients, 10 (53%) had been unsuccessfully treated with more than one chemotherapy regimen.

TABLE 3
Characteristics of the 19 patients enrolled in the study.

| Characteristic | No. of Patients |
|---|---|
| Age, years* | |
| <50 | 2 |
| 50-59 | 6 |
| 60-69 | 5 |
| 70+ | 6 |
| Sex | |
| Female | 12 |
| Male | 7 |
| Race/ethnicity | |
| White non-Hispanic | 13 |
| White Hispanic | 5 |
| Black non-Hispanic | 1 |
| Pathology | |
| Adenocarcinoma | 11 |
| Bronchoalveolar | 3 |
| Squamoous cell | 3 |
| Undifferentiated | 2 |
| Metastasis site | |
| Adrenal | 1 |
| Brain | 3 |
| Liver | 1 |
| Lung | 9 |
| Pleura | 1 |
| Multiple sites† | 4 |
| ECOG performance status | |
| 0 | 4 |
| 1 | 14 |
| 2 | 1 |
| HLA | |
| A1 | 3 |
| A2 | 10 |
| Neither | 6 |

Abbreviation: ECOG, Eastern Cooperative Oncology Goup.
*Mean = 62 years; range 36 to 82 years.
†One pancreas/lung/adrenal; one brain/lung; one lung/adrenal; one liver/lung/T-spine.

Clinical Outcomes. Eighteen patients received a total of 30 courses of vaccine, 90 vaccinations in total (Table 4). Five patients received three full courses, and two patients had two full courses. With the exception of one patient taken off study because a serious adverse event (SAE) occurred after the first vaccination (zero courses completed), the remaining 11 patients had one full course, after which they were taken off study because of disease progression. Four patients experienced SAEs after vaccination, none of which was judged to be vaccine-related.

TABLE 4
Outcomes in the 19 Patients Enrolled on Study.

| Outcome | No. of Patients |
|---|---|
| Courses of vaccine received | |
| 0 | 1 |
| 1 | 11 |
| 2 | 2 |
| 3 | 5 |
| Clinical response | |
| Complete | 0 |
| Partial | 1 |
| Stable disease | 5 |
| Progressive disease | 13 |
| Serious AEs (grade 3 and 4) | |
| Pericardial effusion | 2 |
| Renal Failure | 1 |
| Respiratory failure | 1 |
| AEs (grade 1 or 2) | |
| Rash | 1 |
| Chest pain* | 1 |
| Joint pain | 1 |
| Status† | |
| Alive | 7 |
| Dead | 12 |

Abbreviation: AE, adverse event.
*Chest pain/shortness of breath.
†Alive: median follow-up was 36 months (range, 10 to 40 months); time of death ranged from 1 to 23 months after entry on study.

During the first course of vaccination, a 58-year-old woman developed malignant pericardial effusion requiring a pericardial window; the patient was taken off study, discharged to hospice, and died 1 week later. She had previously been treated unsuccessfully with five lines of palliative chemotherapy before enrollment on study. A 76-year-old male patient also developed a pericardial effusion requiring a pericardial window, but review of prior scans revealed developing pericardial effusion before entry on study. This patient, who had received three courses of vaccine before the SAE developed, continues to have stable disease. He is currently alive and well after 31 months without any further therapy.

A 55-year-old male was found to have worsening of chemotherapy-induced renal dysfunction the day of his first vaccination after he had already signed consent 1 week earlier and underwent a preliminary skin test. His renal function continued deteriorating in the following days, and he died 3 months later. The fourth patient who experienced a SAE was a 56-year-old woman with brain metastasis. During her second course of vaccination, she developed respiratory failure, was then taken off study, and died within 30 days from progression of her disease. This patient had previously been unsuccessfully treated with four lines of palliative chemotherapy.

Regarding other side effects, one patient complained of transient pain at the injection site. Four patients developed some erythema at the vaccination site that resolved within a week. One patient experienced moderate arthritic pain in several joints after the first course. We did not find any patients with significant alteration of their laboratory parameters. including: complete blood and platelet counts, creatinine/BUN, calcium, and liver function tests.

Table 5 shows time to response, duration of response, and survival time for the six patients who had response on study.

TABLE 5
Time to Response, Duration of Response, and Survival
Time for the Six Patients Who Had Response on Study.

| | | | Duration | |
|---|---|---|---|---|
| Patient | | Time to Response | of Response | Survival Time |
| ID | Response | (months) | (months) | (months)* |
| 1010 | PR | 2.3 | 13 | 36+ |
| 1003 | SD | 1.9 | 39+ | 40+ |
| 1004 | SD | 1.6 | 3.5 | 23 |
| 1007 | SD | 2.1 | 2.5 | 37+ |
| 1014 | SD | 2.3 | 3.5 | 21+ |
| 1016 | SD | 1.9 | 1.6 | 11+ |

Abbreviations: PR, partial response; SD, stable disease
*Patients alive as of February, 2004 denoted by plus sign.

One patient had a partial response lasting 13 months, and five showed stable disease ranging from 1.6 to 39+ months (Table 5). The clinical response rate was 32% (six of 19 patients). As of February 2004, these patients had survival times ranging from 23 to 40+ months, and five patients were still alive.

After the patient who had a partial response developed new malignant lesions, verified by positron emission tomography scan, she was put under observation for 2 months because her disease was judged clinically nonaggressive. Several lesions subsequently decreased in size or disappeared. This patient continues to have stable disease without need of palliative chemotherapy 36 months after completing vaccination. Only one of the six patients who had a response on treatment required subsequent palliative chemotherapy. The remaining five patients continue to have stable disease without need of further treatment.

Among the other 13 patients who did not respond to therapy, only two were alive as of February 2004. One of these patients experienced disease stabilization with gefitinib (Iressa™), and the other is undergoing palliative chemotherapy.

Logistic regression analyses of age, sex, race, pathology, and HLA-matching of vaccine showed that none of these factors were statistically significantly related (P>0.10 in all instances) to clinical response (that is, to partial response or stable disease).

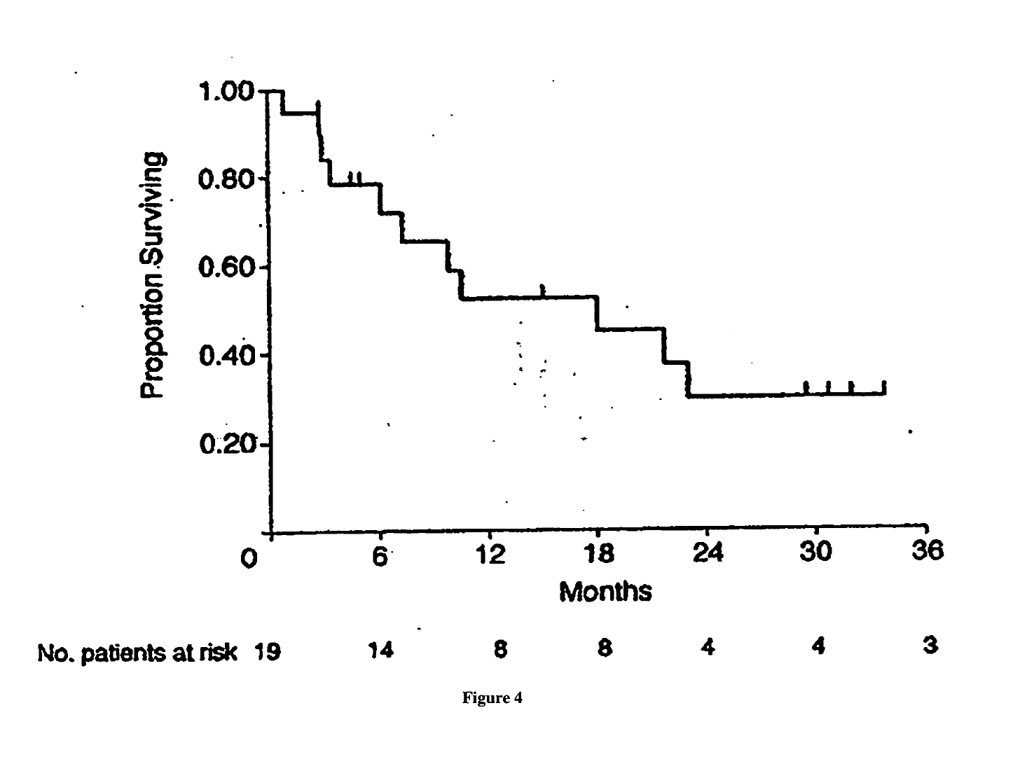

FIG. 4 shows the Kaplan-Meier estimate of overall survival for the 19 study patients (vertical tick marks indicate censored follow-up). The estimated median survival time is 18 months (90% CI, 7 to 23 months). Estimates of 1-year, 2-year, and 3-year overall survival are 52% (90% CI, 32% to 71%), 30% (90% CL 11% to 49%), and 30% (90% CI, 11% to 49%), respectively. As of February 2004, death had occurred in 12 patients from 1 to 23 months after entry on study (Table 2). For the seven patients who are still alive, follow-up from study entry currently ranges from 10 to 40 months, with a median follow-up time of 36 months.

Univariate proportional hazards regression analysis suggested a possibly higher mortality rate in patients receiving HLA-matched vaccine (hazard ratio=4.5; 90% CI, 1.1 to 17.2), and a possibly lower mortality rate in patients with adenocarcinoma (hazard ratio=0.3; 90% CI, 0.1 to 1.0). A multivariate analysis involving five covariates (HLA-matching, age, sex, race, pathology), however, discounted an adverse effect of HLA-matching of vaccine on overall mortality; the corresponding adjusted hazard ratio was 1.9 (P=0.51). The adjusted hazard ratio for adenocarcinoma versus other pathologies was 0.2 (P=0.11), which is within the realm of chance at conventional levels of significance.

Immune Response to Vaccination. This cohort of patients had been heavily pretreated and carried large tumor burdens that are believed to be immunosuppressive. It was important, therefore, to establish whether the tumor vaccination protocol was able to induce a specific immune response in these patients. Since the CD8 CTL response is thought to be critical for tumor rejection, studies were focused on this arm of the immune system. To distinguish between nonspecific natural killer (NK) activity and CD8 CTL activity, a two-fold strategy was employed. First, CD8 cells were purified to eliminate NK cells by including anti-CD56 in the negative selection cocktail of antibodies. Second, the CD8 cells were challenged with K562, an NK target. NK contamination would result in high titers of cells responding to K562 challenge.

All but one patient had a measurable CD8 response after 6 weeks (three vaccinations) that tended to increase after 12 weeks and stabilize by 18 weeks (Table 6). In vitro challenge of patient CD8 cells with wild type A1 or A2 transfected AD100 did not reveal significant differences. Two patients (patient Nos. 1012 and 1019) could not be evaluated immunologically because there was no follow-up sample available for analysis due to early disease progression or adverse events. One patient had only a very modest response, while most other patients showed a strong, highly statistically significant response to vaccination (see pre- and postimmunization titers on challenge with vaccine cells, and lack of response to K562 control; FIG. 5, top panels). All but one patient had a measurable CD8 response after 6 weeks (three vaccinations) that tended to increase after 12 weeks and stabilize by 18 weeks (Table 6). In vitro challenge of patient CD8 cells with wild type A1 or A2 transfected AD100 did not reveal significant differences. Two patients (patient Nos. 1012 and 1019) could not be evaluated immunologically because there was no follow-up sample available for analysis due to early disease progression or adverse events. One patient had only a very modest response, while most other patients showed a strong, highly statistically significant response to vaccination (see pre- and postimmunization titers on challenge with vaccine cells, and lack of response to K562 control; FIG. 5, top panels).

TABLE 6
CDB Response of Vaccinated Patients
Immune Response of CDB Cells to Vaccination*

| | 0 Weeks | | | | 6 Weeks | | | | 12 Weeks | | | | 18 Weeks | | | |
|---|---|---|---|---|---|---|---|---|---|---|---|---|---|---|---|---|
| HLA/Patient | AD- | AD- | AD- | | AD- | AD- | AD- | | AD- | AD- | AD- | | AD- | AD- | AD- | |
| NO. | wt | A1 | A2 | K562 | wt | A1 | A2 | K562 | wt | A1 | A2 | K562 | wt | A1 | A2 | K562 |
| A2/1001 | 4 | 6.2 | 0 | 2.6 | 51 | 49 | 25 | 6 | | | | | | | | |
| A2/1002 | 12 | 19 | 41 | 170 | 30 | 55 | 65 | 96 | | | | | | | | |
| NO/1003 | 1 | 1 | 7 | 0 | 70 | 134 | 53 | 0 | 31 | 113 | 27 | 0 | 49 | 84 | 23 | 6 |
| NO/1004 | 0 | 0 | 0 | 5 | 321 | 424 | 195 | 0 | 216 | 232 | 150 | 0 | 283 | 450 | 130 | 0 |
| A1/1005 | 15 | 0 | 0 | 40 | 92 | 190 | 80 | 34 | | | | | | | | |
| NO/1006 | 13 | 17 | 12 | 11 | 156 | 152 | 132 | 16 | | | | | | | | |
| NO/1007 | 0 | 1 | 0 | 0 | 0 | 3 | 0 | 0 | 1 | 1 | 1 | 0 | 0 | 0 | 2 | 0 |
| NO/1008 | 5 | 6 | 4 | 10 | 97 | 180 | 48 | 3 | | | | | | | | |
| A2/1009 | 3 | 4 | 2 | 17 | 13 | 39 | 13 | 18 | | | | | | | | |
| A2/1010 | 8 | 8 | 4 | 14 | 48 | 87 | 46 | 5 | 120 | 163 | 88 | 8 | 185 | 241 | 157 | 17 |
| A2/1011 | 14 | 20 | 3 | 15 | 80 | 150 | 30 | 12 | 88 | 226 | 57 | 4 | | | | |
| A2/1013 | 18 | 150 | 5 | 0 | 155 | 300 | 164 | 3 | 175 | 154 | 178 | 3 | | | | |
| A2/1014 | 3 | 2 | 2 | 10 | 28 | 20 | 30 | 9 | 30 | 20 | 30 | 12 | 25 | 23 | 25 | 4 |
| A2/1015 | 0 | 0 | 0 | 0 | 0 | 0 | 0 | 0 | | | | | | | | |
| A1/1016 | 138 | 120 | 128 | 4 | 144 | 150 | 163 | 5 | 127 | 120 | 164 | 15 | | | | |
| A2/1017 | 0 | 11 | 0 | 4 | 100 | 200 | 200 | 3 | | | | | | | | |
| NO/1018 | 13 | 44 | 0 | 9 | 51 | 200 | 52 | 9 | | | | | | | | |

NOTE.
CD8 cells challenged at a ratio of 20:1 = CD8:tumor cell. The mean spot number of quadruplicate values is given.
Abbreviations: AD-wt, AD100 untransfected; AD-A1 or AD-A2, AD100 transfected with HLA A1 or A2; HLA NO, No HLA A1 or A2.
*Values are number of interferon-gamma secreting cells (spots per 20,000 CD8 cells) after in vitro challenge.

There was no statistically significant difference in the CD8 response depending on whether or not the patients were HLA-matched to the vaccine (Table 6). Most patients before vaccination had only low or absent immune response to vaccine cells, and equally low activity to challenge with K562. One patient (No. 1016) had strong prevaccination CD8 activity toward AD100 and only minimal activity toward K562 (FIG. 5, last panel), suggesting preexisting immune activity toward the tumor. Another patient (No. 1002) had high prevaccination K562 reactivity of his CD8 cells and low activity toward AD100. Vaccination increased reactivity toward AD100 and tended to decrease CD8 reactivity toward K562 when it was present.

The immune response of the six clinically-responding patients (FIG. 5B, lower panels) shows that CD8 titers to AD100 stimulation continue to be elevated up to 150 weeks after cessation of vaccination.

Given the advanced stage of disease in patients enrolled in the studies disclosed herein, the evidence of some clinical benefit was unexpected and encouraging. Moreover, since the B7-vaccine tested here induced CD8 CTL responses, it may be that the CD8 response is causally related to the clinical outcome seen here. Additional studies are performed in the setting of minimal disease. Patients with early stage NSCLC (stage I/II) are vaccinated after surgery to decrease the chance of relapse and potentially prolong survival.

The results described in this example show that tumor progression can be slowed by vaccination and that this effect occurs regardless of whether or not patients are allogeneic to the HLA A1 or A2 locus of the vaccine. These findings also support indirect antigen presentation as being effective in promoting antitumor activity and that allogeneic MHC molecules enhance the effect.

Throughout this application various publications have been referenced. The disclosures of these publications in their entireties are hereby incorporated by reference in this application in order to more fully describe the state of the art to which this invention pertains to the same extent as if each was specifically and individually indicated to be incorporated by reference. The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

1. A method for stimulating an immune response against a non-immunogenic tumor, comprising administering to a subject in need thereof an immunogenic amount of a tumor cell line genetically modified to express a nucleic acid encoding CD80 and a nucleic acid encoding a human leukocyte antigen (HLA).

2. The method of claim 1, wherein the non-immunogenic tumor results from any sarcoma or carcinoma.

3. The method of claim 1, wherein the non-immunogenic tumor results from lung cancer.

4. The method of claim 1, wherein the lung cancer is non-small cell lung cancer (NSCLC), squamous cell carcinoma, large cell lung cancer, adenocarcinoma of the lung, bronchial carcinoma, or small cell lung cancer.

5. The method of claim 1, wherein the non-immunogenic tumor results from colo-rectal cancer.

6. The method of claim 1, wherein the non-immunogenic tumor results from pancreatic cancer.

7. The method of claim 1, wherein the non-immunogenic tumor results from head and neck cancer.

8. The method of claim 1, wherein the tumor cell line is allogeneic.

9. The method of claim 1, wherein the tumor cell line is a tumor cell from lung cancer.

10. The method of claim 1, wherein the tumor cell line is a line named AD100.

11. The method of claim 1, wherein the human leukocyte antigen (HLA) is HLA-A1, HLA-A2, HLA-A3, or HLA-A27.

12. The method of claim 1, wherein the immunogenic amount of the tumor cell line is administered in a unit dosage range of between 1×10⁶ and 1×10⁸ cells.

13. The method of claim 1, wherein the unit dosage is about 5×10⁷ cells.

14. The method of claim 1, wherein the tumor cell line is administered bi-weekly.

15. The method of claim 1, wherein the tumor cell line is administered every four weeks.

16. The method of claim 1, wherein the tumor cell line is administered every six weeks.

17. The method of claim 1, wherein the tumor cell line is administered every eight weeks.

18. The method of claim 1, wherein the administering step is performed orally, transdermally, or nasally.

19. The method of claim 1, wherein the administering step is performed by parenteral or intradermal injection.

20. The method of claim 1, wherein the parenteral injection is subcutaneous or intramuscular.

21. The method of claim 1, wherein the immune response is indicated by an increase in the frequency of interferon-γ secreting CD8 T cells of the subject.

22. The method of claim 1, wherein the immune response is indicated by an increase in the frequency of tumor necrosis factor-alpha secreting CD8 T cells of the subject.

23. The method of claim 1, wherein the immune response is indicated by an increase in the frequency of interleukin-2 secreting CD8 T cells of the subject.

24. The method of claim 1, further comprising administering an antibody.

25. The method of claim 1, further comprising administering radiation therapy or chemotherapy, or performing surgery.

26. The method of claim 1, wherein the tumor cell line is administered within six weeks following chemotherapy.

27. The method of claim 1, wherein the tumor cell line is administered within four weeks following surgery.

28. The method of claim 1, wherein the tumor cell line is formulated as a vaccine.

29. The method of claim 1, further comprising one or more booster immunizations.

30. A method for stimulating an immune response against a non-immunogenic tumor, comprising administering to a subject in need thereof an immunogenic amount of a tumor cell line genetically modified to express a nucleic acid encoding a co-stimulatory factor and a nucleic acid encoding a major histocompatibility complex protein.

31. The method of claim 30, wherein the co-stimulatory factor is selected from the group consisting of CD80 and CD86

32. The method of claim 30, wherein the major histocompatibility complex protein is selected from the group consisting of: HLA-A1, HLA-A2, HLA-A3, and HLA-A27.

33. An immunogenic composition suitable for administration to a human, comprising:
a population of allogeneic tumor cells comprising at least two allogeneic tumor cells, each of which is genetically altered to express a nucleic acid encoding CD80 and a nucleic acid encoding a human leukocyte antigen (HLA), wherein the composition is effective to elicit an immune response against a non-immunogenic cancer in the human after administration.

34. A unit dose of the immunogenic composition according to claim 33, wherein the tumor cell population in the dose ranges from about 1×10⁶ and 1×10⁸ cells.

35. A unit dose of the immunogenic composition according to claim 33, wherein the tumor cell population includes about 5×10⁷ cells.

36. A method for producing an immunogenic composition, comprising:
providing a population of allogeneic cells;
transfecting the population of cells with plasmid cDNA encoding CD80 and encoding a human leukocyte antigen (HLA).

37. A pharmaceutical composition comprising an immunogenic amount of a tumor cell line genetically modified to express a nucleic acid encoding CD80 and a nucleic acid encoding a human leukocyte antigen (HLA), and a pharmaceutically acceptable excipient, carrier, or diluent, wherein the composition is effective to elicit an immune response against a non-immunogenic cancer in a subject after administration.

38. The pharmaceutical composition of claim 37, wherein the non-immunogenic cancer is any sarcoma or carcinoma.

39. The pharmaceutical composition of claim 37, wherein the non-immunogenic cancer is lung cancer.

40. The pharmaceutical composition of claim 37, wherein the lung cancer is non-small cell lung cancer (NSCLC), squamous cell carcinoma, large cell lung cancer, or small cell lung cancer.

41. The pharmaceutical composition of claim 37, wherein the non-immunogenic cancer is colo-rectal cancer.

42. The pharmaceutical composition of claim 37, wherein the non-immunogenic cancer is pancreatic cancer.

43. The pharmaceutical composition of claim 37, wherein the non-immunogenic cancer is head and neck cancer.

44. The pharmaceutical composition of claim 37, wherein the tumor cell line is allogeneic.

45. The pharmaceutical composition of claim 37, wherein the tumor cell line is a tumor cell from lung cancer.

46. The pharmaceutical composition of claim 37, wherein the tumor cell line is AD100.

47. The pharmaceutical composition of claim 37, wherein the human leukocyte antigen (HLA) is HLA-A1, HLA-A2, HLA-A3, or HLA-A27.

48. The pharmaceutical composition of claim 37, wherein the immunogenic amount of the tumor cell line is administered in a unit dosage range of between 1×10⁶ and 1×10⁸ cells.

49. The pharmaceutical composition of claim 37, wherein the unit dosage is about 5×10⁷ cells.

50. The pharmaceutical composition of claim 37, wherein the tumor cell line is administered bi-weekly.

51. The pharmaceutical composition of claim 37, wherein the tumor cell line is administered every four weeks.

52. The pharmaceutical composition of claim 37, wherein the tumor cell line is administered every six weeks.

53. The pharmaceutical composition of claim 37, wherein the tumor cell line is administered every eight weeks.

54. The pharmaceutical composition of claim 37, wherein the administering step is performed orally.

55. The pharmaceutical composition of claim 37, wherein the administering step is performed by parenteral or intradermal injection.

56. The pharmaceutical composition of claim 37, wherein the parenteral injection is subcutaneous or intramuscular.

57. The pharmaceutical composition of claim 37, wherein the immune response is indicated by an increase in the frequency of interferon-γ secreting CD8 T cells of the subject.

58. The pharmaceutical composition of claim 37, wherein the immune response is indicated by an increase in the frequency of tumor necrosis factor-alpha secreting CD8 T cells of the subject.

59. The pharmaceutical composition of claim 37, wherein the immune response is indicated by an increase in the frequency of interleukin-2 secreting CD8 T cells of the subject.

60. The pharmaceutical composition of claim 37, further comprising an antibody.

61. The pharmaceutical composition of claim 37, wherein the composition is administered in conjunction with radiation therapy, surgery, or chemotherapy.

62. The pharmaceutical composition of claim 37, wherein the tumor cell line is administered within six weeks following chemotherapy.

63. The pharmaceutical composition of claim 37, wherein the tumor cell line is administered within four weeks following surgery.

64. The pharmaceutical composition of claim 37, wherein the composition is formulated as a vaccine.

65. The pharmaceutical composition of claim 37, wherein the vaccine additionally contains an adjuvant or an immunomodulator.

66. The pharmaceutical composition of claim 37, wherein the composition is formulated as a booster immunization.

67. A pharmaceutical composition comprising an immunogenic amount of a tumor cell line genetically modified to express a nucleic acid encoding a co-stimulatory factor and a nucleic acid encoding a major histocompatibility complex protein, and a pharmaceutically acceptable excipient, carrier, or diluent, wherein the composition is effective to elicit an immune response against a non-immunogenic cancer in a subject after administration.

68. The pharmaceutical composition of claim 67, wherein the co-stimulatory factor is selected from the group consisting of CD80 and CD86

69. The pharmaceutical composition of claim 67, wherein the major histocompatibility complex protein is selected from the group consisting of: HLA-A1, HLA-A2, HLA-A3, and HLA-A27.
